(19) United States
(12) Patent Application Publication
(10) Pub. No.: US 2022/0257479 A1
(43) Pub. Date: Aug. 18, 2022

(54) Gynecological vaginal wash for adjusting female vaginal microecology and preparation method thereof

(71) Applicant: NANOLATTIX BIOTECHNOLOGY CO., LTD., Taiyuan, Shanxi (CN)

(72) Inventors: Shaoping Li, Taiyuan, Shanxi (CN); Li Shaoping, Taiyuan, Shanxi (CN)

(21) Appl. No.: 16/338,691

(22) PCT Filed: Mar. 8, 2018
(86) PCT No.: PCT/CN2018/078433

§ 371 Date: Sep. 14, 2020

(30) Foreign Application Priority Data
Mar. 10, 2017 (CN) 201710140896.7

(51) Int. Cl. A61K 8/19; A61K 8/97; A61Q 19/00

(52) U.S. Cl. CPC A61K 8/19; A61Q 19/00; A61K 8/97

(57) ABSTRACT

A gynecological vaginal wash for adjusting female vaginal microecology and a preparation method thereof, The wash is prepared by adding a natural fragrance to acidic water with an acidic water volume concentration of 0.01-0.5% and adjusting a pH value thereof to 3.5-5.5 after disinfection.

### TECHNICAL FIELD

The present invention belongs to the field of health care and medical technology, and particularly is a gynecologic vaginal spray for regulating female vaginal microecology and the preparation method thereof.

### BACKGROUND ART

As we all know, the female reproductive tract is an open cavity that is an important micro-ecological area of the human body. This area is a microecological system mainly composed of bacteria. An imbalance in the female vaginal microecology is the main cause for gynecologic vulvar and vaginal inflammation. Among the factors that maintain the vaginal microecological balance, estrogen, lactobacilli and vaginal pH play a vital role. Under normal physiological conditions, estrogen thickens the vaginal epithelium and is rich in glycogen, which enhances the resistance to pathogens. Glycogen is decomposed into lactic acid by the vaginal lactobacillus to maintain normal vaginal acidic environment (pH ˜4.5, usually between 3.8 to 4.4), to inhibit the growth of other parasites and pathogens, which is the process of vaginal self-cleansing.

The study of vaginal microecology is mainly focused on vaginal normal flora. As early as 1892 Dederlein first published the study on the human vaginal microecological flora. Later, it was found that the vaginal flora of healthy women is composed of a variety of anaerobic and aerobic bacteria. So far, twenty-nine types of microorganisms have been isolated from vaginal secretions, the most important of which is Lactobacillus, which is found in 50% to 80% of vaginal secretion specimens of healthy women. Lactobacillus produces a number of antimicrobial substances, including lactic acid, bacteriocin, lactose B and hydrogen peroxide. Hydrogen-peroxide producing Lactobacilli play an important role in controlling the vaginal microecological environment and inhibit the overgrowth of other pathogens by combined action with other peroxides.

In women of normal childbearing age, there is sufficient estrogen in the body, together with dominant lactobacillus in the vagina, to maintain an acidic vaginal environment (pH is about 3.8-4.5). Under normal physiological conditions, vaginal epithelial cells change cyclically with menstruation under the control of estrogen and progesterone, especially the surface cells, which are rich of glycogen. After glycogen is secreted, glycogen is decomposed into lactic acid by vaginal bacilli, so that the vagina is kept at pH 4.5 acidic environment, thereby inhibiting the the reproduction of pathogenic bacteria; hence the healthy vaginal fluids render a self-purification or sterilization ability.

When the reproductive tract is inflamed or pH increases, the vaginal environment changes, and the number of pathogenic bacteria and white blood cells present increases. According to the presence of vaginal bacilli in the vaginal fluid, as well as the number of pathogenic bacteria and white blood cells, the degree the cleanliness of the vaginal fluid is called determined. Suggestions: the normal pH of vaginal secretions is 3.8. When the pH is 3.8˜4.2, it is conducive to the reproduction of symbiotic bacteria, especially lactobacilli, which is the main bacterium in the health vagina. Vaginal fluid density can be up to 105˜108 ml. When the vagina is infected by microorganisms, if lactobacillus is dominant, the vagina can still maintain pH3 0.8˜4.2, without succumbing to disease, as the lactobacillus can produce H2O2, which is toxic to other microorganisms and inhibits their reproduction.

The other bacteria, such as Streptococcus suis, Enterobacter, Proteus, Gaffkya anaerobia, and Veillonella are common in the lower part of the vagina, and usually do not cause symptoms. The vaginal bacteria work together restricting and balancing each other and inhibiting pathological bacteria from having an effect; if this balance is disrupted and the mutual restrictions removed, the concentration of hydrogen ions decreases, Lactobacillus loses its dominant role, and pathological bacteria can multiply. Bacterial vaginosis arises due to the reduction of vaginal lactobacilli and the growth of other bacteria, mainly mixed infections caused by Gardnerella, various anaerobic bacteria and mycoplasma.

The resulting acidic environment caused by Lactobacillus is conducive to reducing the negative charge on the cell surface and removing the glycosyl groups that cover the surface of the receptor, thereby exposing the receptor and helping the bacteria to adhere. Therefore, lactobacilli regulate the normal vaginal flora through both pros and cons. Once vaginal micro-ecological balance is broken or foreign pathogens invade, inflammation can occur, such as estrogen level changes or vaginal pH increases; abuse or use of antibiotics will make bacteria resistant, and kill a large number of beneficial bacteria, destroy the vaginal flora balance. A vaginal flora imbalance will lead to the growth of fungi and tenacious gynecological diseases that are difficult to completely overcome.

For example, trichomonas vaginitis, which is caused by Trichomonas vaginalis, thrives when the pH is between 5.2 to 6.6. Hence, many women who have this gynecological disease are often seen clinically, because the disease relapses cyclically with menstruation.

It can be seen that the vaginal pH is very important, including in the prevention of gynecological diseases. The vaginal pH is weakly acidic. The weak acidic pH reveals the truth that washing the vagina with alkaline substances tends to destroy the weakly acidic vaginal environment and is good for bacterial reproduction. The pH of the vagina is acidic. Normal vaginal pH≤4.5, mostly in 3.8-4.4. When suffering from vaginal inflammation, the pH of the vagina will change, where the change in pH is closely correlated to the pathogens causing the inflammation. Different pathogens lead to different degrees of inflammation and different changes in pH value.

After menstruation and sexual activity, vaginal pH will deviate from the normal range, and is conducive to bacterial reproduction.

In summary, improving the vaginal microenvironment, repairing vaginal micro-injuries, maintaining vaginal micro-ecological balance, improving vaginal cleanliness, and improving vaginal mucosal immunity, are very important for the treatment of common vaginal disease in women. Avoiding the use of antibiotics should be considered due to drug resistance, dual infection, allergic reactions and other side effects. At present, most gynecological gels contain Chinese traditional medicine ingredients, and they are mainly used to treat gynecological diseases by inhibiting harmful bacteria. But, killing the pathogens also kills many probiotics at the same time, resulting in imbalance of vaginal flora. The vaginal immunity is low, which leads to recurrent, incurable vaginitis.

Therefore, how to develop a reasonable adjuvant therapy that can maintain the female vaginal microecological balance, so as to prevent and treat the female vaginal microecological imbalance, is an important challenge for scientific researchers and medical workers.

Electrolyzed functional water is the general term for acidic water and alkaline water with special function made from a special device where the electrolyte solution is treated by the electric field to change its pH, oxidation-reduction potential (ORP), effective chlorine concentration (ACC) and other properties of the water. According to its physical and chemical properties, electrolyzed functional water can be divided into electrolytic strong acid functional water, electrolytic slightly acid functional water, electrolytic weakly alkaline functional water and electrolytic alkaline ionized water.

Acidic oxidation potential water is the general term for all kinds of artificial electrolysis strong functional water, which includes electrolytic strong acidic water, electrolytic weak acidic water, ionized acidic water and artificial hydrogen-rich nano-bubble acidic water. The pH of artificial electrolysis strong acid water is usually between 2.0 to 3.5, with a higher oxidation-reduction potential, generally up to 1000˜1200 mV, effective chlorine concentration of 20˜200 mg/L. The artificial electrolytic acid water (including strong acidic water and slight acidic water) has disinfection and sterilization function, mainly for the preservation of fruits and vegetables, sprouts productions, fresh cut flowers, equipment disinfection, toxins degradation, animal husbandry and plant protection fields. The sterilization mechanisms of artificial electrolytic acidic water are mainly free radical theory, effective chlorine theory, and strong oxidation theory of oxidation-reduction potential.

The pH of the artificial electrolytic weak acidic water is usually between 3.5 and 6.0. Generally the artificial electrolytic acidic water is defined as: a water solution with less than 0.2% of NaCl that is electrolyzed in an electrolytic cell separated by a membrane and Hypochlorous acid (HClO) that is the main active ingredient obtained from the cathode. Artificial weak electrolytic acidic water can be consumed, used for cleaning or as disinfectant. Artificial strong electrolytic acidic water is used in the industrial and medical field as industrial disinfectant for equipment, etc. Experts hypothesized that artificial electrolytic acidic water contains certain concentration of hypochlorous acid (HOCl or HClO) that causes a decrease in pH and the OCl— in the hypochlorous acid produces bactericidal effect.

Ionized water is filtered tap water through the activated carbon as a filter layer. The tap water is filtered and purified to the national criterion. Diaphragm electrolysis is then used to generate two kinds of active water or ionized water. The ionized alkaline water is produced from anode, and the ionized acidic water is produced from cathode.

Essentially ionized water is still water with no addition of any material only loss/gain of electrons. The ionized water will gradually reduce into ordinary water by contacting with air or other substances during usage. The ionized water is produced by ionization. The tap water is purified by high efficiency ionization tank, and through high-tech means, it is separated into positive and negative ionized water. Ionized water differs from ordinary water in terms of solubility, conductivity, pH, surface tension, density, thermal conductivity, and oxygen content, etc. So, ionized water is more active than ordinary water (including mineral water, pure water)

It is generally believed that the ionized acidic water contains hydrogen ions (H4O9+), which produce low pH acidic water, also known as oxidized water. The unique functions of the ionized water, such as beauty aid, anti-inflammatory and sterilization, are not found in ordinary water. Its usages in industrial and agricultural field are still being developed.

Nano-bubble hydrogen-rich acidic water contains a large amount of nano-bubble hydrogen-rich bubbles. It has contains more hydrogen than ordinary hydrogen water. The hydrogen concentration is about 1/20 or more of the saturated concentration.

Air bubble occurs when gas is present in liquid solutions. In nature, different sizes and shapes of bubbles will form in liquid when gas meets with a shear force. Currently, the classification and definition of bubbles is not very strict. According to the size of the bubble, it can be classified into centimeter bubble (CMB), millimeter bubble (MMB), micron bubble (MB), micro-nanobubble (MNB), nano bubble (NB).Micro-nano bubbles are about 10 microns to hundreds of nanometers in diameter and between the micro-bubble and nano-bubbles. This kind of bubbles has its unique physical and chemical properties.

### Micro-Nano-Bubble Characteristics

1. Large specific surface area. The relationship between the volume of the bubbles and the surface area can be expressed by the following formulas. The volume formula of the bubble is V=π/3r3, the surface area of the bubble is A=4π2, and the two formulas are combined to obtain A=3V/r, that is, V=n·A=3V total/r. The total surface area of the bubbles is inversely proportional to the diameter of the individual bubbles in the case of the total volume is unchanged (V constant).According to the formula, the specific surface area of the former is 10 times higher than that of the 1 mm bubble in a volume of 10 microns. The contact area of air and water increased by 100 times, a variety of reaction speed also increased by 100 times.

2. Slow rising speed. According to Stokes' law, the rising rate of air bubbles in water is proportional to the square diameter of the bubble. The smaller the diameter, the slower the rising speed is. From this relationship, it concludes that an air bubble with a diameter of 1 mm will have a rising speed of 6 m/min and an air bubble of 10 um will have a rising speed of 3 mm/min. The latter is 1/2000 of the former. And if you take into account the larger surface area, then the solubility of the micro-nano-bubble is 200,000 times of the common air.

3. Surface charge: pure water solution is composed of water molecules and a small amount of ionization generated H+ and OH— composition, the formation of air bubbles in the water-gas interface is easy to accept the characteristics of H+ and OH—, and usually cation is away from the water-gas interface more easily than the anion, leaving the interface often with a negative charge. The surface that has been charged generally tends to adsorb counterions in the medium, especially the expensive counterions, to form a stable double electric layer. The potential difference of the surface charge of the microbubbles is often characterized by the zeta potential, and the zeta potential is an important factor in determining the adsorption performance at the bubble interface. When the micro-nano bubbles shrink in water, the charge ions are rapidly enriched at very small bubble interfaces, showing a significant increase in zeta potential, and a very high zeta potential can be formed at the interface before the bubble burst.

4. Self-pressurized dissolving: There is a gas-liquid interface around the bubbles in the water, and the presence of the gas-liquid interface causes the bubbles to be affected by the surface tension of the water. The bubbles with a spherical interface, the surface tension compresses the gas in the bubble, allowing more gas in the bubble to dissolve into the water. According to the Young-Laplacian equation, ΔP=2σ/r, ΔP represents the value of the pressure rise, σ represents the surface tension, and r represents the bubble radius. The pressure of the bubble having a diameter of 0.1 mm or more is negligible but bubble having a diameter of 10 um is subjected to a pressure of 0.3 atm, and the bubble having a diameter of 1 um is subjected to a pressure of up to 3 atm. The dissolution of micro-nano-bubbles in water is a process in which bubbles gradually shrink, and the increased pressure increases the rate of gas dissolution. With the increase of specific surface area, the rate of bubble shrinkage becomes faster and faster, and finally dissolves into water. Theoretically, the pressure exerted on the bubble can be infinite.

5. Generation of large amount of free radicals: Due to the disappearance of gas-liquid interface during the micro-bubble rupture, the highly concentrated ions on the interface surface released the stored chemical energy at once. This process stimulates a larger amount of hydroxyl radicals. Hydroxyl radicals have ultra-high oxidation and reduction potential. This can oxidize difficult decomposition of pollutants in water such as phenol and thus purify the water.

Micro bubbles and nano bubbles have the ability to break polymer molecules into small active water molecules, can cause a small part of the water molecules ionized decomposition, and can generate reactive oxygen, oxygen ions, hydroxyl ions and other free radicals, especially hydroxyl radicals have ultra-high reduction potential, with super-oxidation effect, so can decompose pollutants that are difficult to be decomposed under normal conditions. The dissolution rate of the micro-nano-bubbles in water is more than 85%, the dissolved oxygen concentration can reach above the saturation concentration, and the micro-nano bubbles are stored in water in the form of bubbles for a long time (increase speed 6 cm/min), which can be dissipated with dissolved oxygen constantly replenishing reactive oxygen to the water, providing sufficient reactive oxygen and strong oxidizing ionic groups for microorganisms to treat sewage, and ensuring a sufficient reaction time for reactive oxygen. The ability to purify water treated by micro-nano bubbles is much higher than self - purification in natural conditions.

The contents of the invention

This invention, unlike traditional gynecological lotion, does not kill probiotics while killing pathogens. While the traditional gynecological lotion can lead to vaginal dysplasia, vaginal immunity, and cause recurrent episodes of vaginitis, this invention provides a special nanowater for female vaginal system health and its preparation method

The invention is achieved by the following technical method: this gynecological vaginal lotion that regulates the female vaginal microecology is acidic oxidative potential water with 0.01% to 0.5% of natural spices and adjusted to pH 3.5-5.5 after disinfection.

Acidic oxidation potential water may be a mixture of nano-bubbles hydrogen-rich acidic water and electrolytic acidic water. The hydrogen concentration is 1-50 ppm, and the oxidation-reduction potential OR is from −400 mV to 1200 my, the pH of nano-bubbles hydrogen-rich acidic water is 4.5 to 7.5, and the pH of the electrolytic acidic water is 2.0-4.5.

Acidic oxidation potential water can also be pH 3.8 to 5.5 electrolytic acidic water with 0.1% to 0.5% acid salt containing mineral acid. The effective chlorine concentration is ≥8 ppm, and LD5t1 is greater than 300 mg. Acid salt containing hydrogen atom that is capable to be substituted with a metal atom or is acidic with pH less than 7.0 in water.

Electrolyzed acidic water is prepared by electric field using natural water or tap water added less than 0.5% salt.

Acidic oxidation potential water may also be a mixture of electrolytic acidic water and artificial ionized acidic water. Artificial ionized acidic water is natural tap water purified through high efficiency ionization tank so that the water is ionized and separated into positive and negative ionized water.

Acidic oxidation potential water is prepared by mixing nano-bubble hydrogen-rich acidic water and artificial electrolytic acidic water in any proportion.

Acidic oxidation potential water is prepared by mixing electrolytic acidic water and artificial electrolytic acidic water in any proportion.

Inorganic acid salt is an inorganic acid sodium salt, inorganic acid potassium salt, inorganic acid magnesium salt, or inorganic acid barium salt. The pH for these inorganic acid salts is between 3.5-6.0.

Inorganic acid salt is any mixture of dihydrogen disodium pyrophosphate, sodium hexametaphosphate, sodium chloride and sodium dihydrogen phosphate.

The pH of the female vaginal lotion is 3.8-5.0 and it is specifically made for mature women.

The pH of the modified vaginal lotion for young girls is 4.5-5.5.

The natural spices in the lotion are botanical natural spices such as rose oil, jasmine extract, vanilla tincture, brandy balsam, turmeric resin, and narcissus net oil.

One preparation method for Nano-bubble hydrogen-rich acidic water is: (1) degassing: the premix storage tank is vacuumed and pure water is stirred in order to remove the gas from the pure water; (2) the first mixture of gas and water: the pre-mixed storage tank is filled with hydrogen gas and the pure water is stirred to make the hydrogen gas and the purified water premixed to obtain the first gas-water mixture; (3) the second gas-water mixture: deliver the step (2) water mixture to the secondary gas-liquid mixed chestnut, and fill the secondary gas-liquid mixed chestnut with hydrogen, so that the mixture of gas and water is mixed with hydrogen further to form the second gas-water mixture; (4) Increasing pressure: Adding the secondary gas-water mixture obtained in the step (3) into the pressure tank to pressurize the mixed gas-water mixture; (5) passing the pressurized secondary air-water mixture into the aeration head to produce nano-bubble hydrogen-rich acidic water.

Electrolytic acidic water is prepared by adding 0.1% to 1% mineral acid salt. Then, electrolyze it in a membrane electrolytic cell, and select the electrolytic acidic water in the anode of the electrolytic cell. The pH of the acidic water is from 2.0 to 4.0.

Artificial ionization acidic water preparation: tap water is filtered by activated carbon, and is then electrolyzed through a membrane electrolytic cell. Solution on the anode of the electrolysis tank is acidic nano-ion water. Depending on the size of the electrolysis equipment, the size of the electrolytic current ranges from a few milliamps to several amps, and the ionization electrolysis time ranges from 1 minute to 30 minutes.

Nano-bubble hydrogen-rich water, electrolytic acidic water, artificial electrolytic acidic water and artificial ionized acidic water can also be purchased directly from the market.

The gynecological vaginal health liquid or the medicinal gynecological lotion can treat women fungal, bacterial, trichomonas vaginitis and cervical erosion disorder. The gynecological lotion has a strong adhesion with the mucous membrane, which allows it to adhere to the vagina for a longer time thus improving the efficacy. Irritation to the skin and mucous membranes is weak because the pH of the gynecological lotion is 3.8 - 5.5.

Female gynecological maintenance at pH 4.0-5.5. This weak acidic environment can effectively maintain the cleanness of genitalia. This pH is different for women of different ages, 4.5-5.5 for young girls and 3.8-5.0 for mature women.

The acidic water in this invention can regulate pH balance, correct the flora, and insecticidal effect, and has the advantages of convenient administration, good retention to the vagina, no stimulation, no foreign body sensation and no damage to the vagina. This invention is an aqueous solution which greatly improves the bioavailability; the invention has the good therapeutic effect on the most common bacterial vaginosis infection in the gynecological reproductive tract, and the invention does not contaminate the clothing.

This product can effectively improve the vaginal cleanliness, improve the number of lactobacilli, reduce the vaginal pH, and repair vaginal. It can be used as an adjuvant therapy for vaginitis and cervicitis, and has no side effects, such as no damage on liver and kidney, and hematuria routine examination did not find abnormal changes. This indicates that the use of this invention is safe.

Compare to other products, the advantages of this invention are that it balances the pathogenesis of female vaginal microecological by inhibiting pathogen and promoting the proliferation of Lactobacillus. The related products on the market are aimed at sterilization, which results in the destruction of the normal flora balance. The formula of the invention increases the absolute number of dominant bacteria Lactobacillus and balances its growth at 60% or greater, while reducing the number of pathogens to maintain vaginal micro-ecological balance. This product improves vaginal cleanliness, vaginal mucosal immunity, prevents of gynecological vaginal inflammation, and reduces the recurrence rate of gynecological vaginitis. Compare to other products such as fungicides, this product is more safe and secure to use.

The invention adopts the optimized preparation technology, determines the reasonable proportion of the raw material and improves the regulating performance of the vaginal pH. This product of gynecological vaginal lotion has high absorption rate, stable quality, and no side effects. By scientific preparation, this acidic oxidation potential water can last for a long time, easy to be completely absorbed, can make nanowater diffusion in molecular level, greatly improve the acidic oxidation potential water's bioavailability.

### BRIEF DESCRIPTION OF THE SEVERAL VIEWS OF THE DRAWINGS

FIG. 1. A model depicting the bubble size in solution.

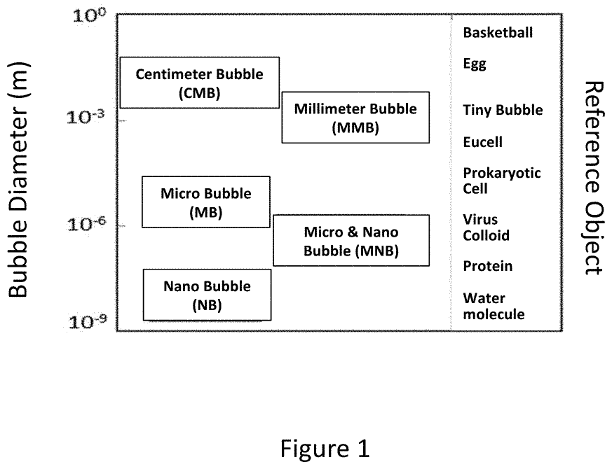

FIG. 2. A model depicting the charge characteristics of the micro-nano bubble surface.

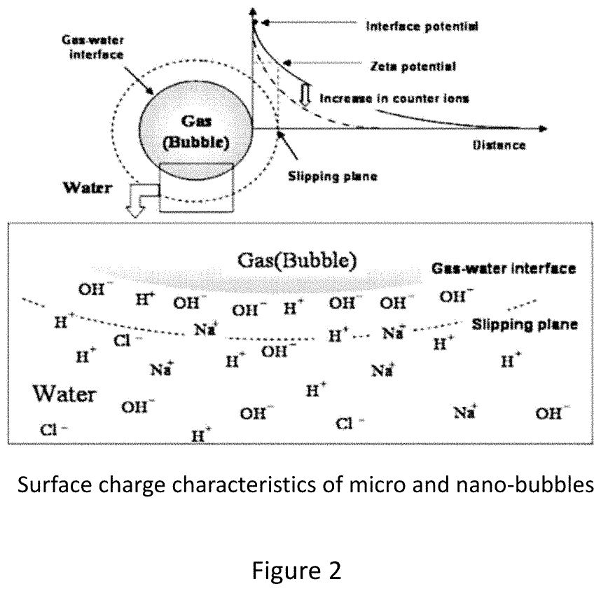

FIG. 3. A model depicting the preparation of the artificial electrolytic acidic water.

FIG. 4. A model depicting the equipment for the artificial electrolytic acidic water preparation.

### DETAILED DESCRIPTION OF THE INVENTION

Example 1: A gynecological vaginal lotion for regulating female vaginal microecology, adding natural spice rose oil with a volume fraction of 0.01% of nano-bubble rich water in water-rich water, and adjusting the pH value to 3.5-5.5 after disinfection.

The degassing: the premix storage tank vacuum and pure water to stir, remove the pure water within the gas; (2) a mixture of gas and water: to the pre-mixed storage tank is filled with hydrogen gas and the pure water is stirred to make the hydrogen gas and the purified water premixed to obtain a gas-water mixture; (3) the second gas-water mixture: the step (2) Water mixture to the secondary gas-liquid mixed chestnut, and to the secondary gas-liquid mixed chestnut filled with hydrogen, so that the mixture of fresh water mixed with hydrogen and further mixed to form a second gas-water mixture; (4): Adding the secondary gas-water mixture obtained in the step (3) into the pressure tank to pressurize the mixed gas-water mixture; (5) passing the pressurized secondary air-water mixture into the aeration head to produce nano-bubbles Hydrogenic acid water;

Said hydrogen peroxide having a hydrogen content in the range of 1 to 50 ppm; the oxidation-reduction potential OR is from -400 my to 1200 my; and the pH value of the nano-bubble rich in hydrogenated water is 4.0 to 7.5. Prepared by the regulation of female vaginal microecological gynecological vaginal lotion pH value of 3.8-5.0, for mature women use. Prepared by the regulation of female vaginal microbial gynecological vaginal lotion applied to the pH value of 4.5-5.5, for girls to use.

The nanometer bubble rich hydrogenated water of the present invention is water containing a large amount of nano-bubbles rich in hydrogen bubbles, and contains water having a higher hydrogen concentration than ordinary hydrogen water. The hydrogen concentration is about 1/20 or more of the saturated concentration.

The hydrogenated water of the nano-bubbles contains the hydrogen concentration of 1˜50 ppm and the oxidation-reduction potential of the nano-bubble hydrogen-rich water is ˜400˜1200 mv. The purity of hydrogen can reach more than 99.9999%, in full compliance with the national standard of the People's Republic of China food safety standards for food safety standards for hydrogen.

Example 2: A gynecological vaginal lotion for the regulation of female vaginal microecology, a natural perfume jasmine extract containing 0.05% by volume of electrolytic acidic water, adjusted to pH 3.5-5.5 after disinfection.

Electrolytic acidic water The composition comprises an acid salt containing a mineral acid having a mass concentration of 0.1% to 1%, an effective chlorine concentration in an acid salt containing a mineral acid of ≥8 ppm, and an LD5t1 value of more than 300 mg; A salt of a hydrogen atom capable of being substituted with a metal atom, or an acid salt is an acid salt having a characteristic that the acidity is dissolved in water, i.e., a pH of less than 7.0.

The acid salt of the inorganic acid is an acid sodium salt of an inorganic acid, an acid potassium salt of an inorganic acid, an acid magnesium salt of an inorganic acid, and any one of an acid barium salt of an inorganic acid, For 3.5-6.0.

It is preferable that the acid salt of the inorganic acid is any one or any one of any two or two of sodium dihydrogen phosphate pyrophosphate, sodium hexametaphosphate, sodium chloride and sodium dihydrogen phosphate in any ratio from the mixture.

The regulation of female vaginal microbial gynecological vaginal lotion pH value of 3.8-5.0, for mature women.

The modified vaginal microbial gynecological vaginal lotion is applied to a pH of 4.5 to 5.5 for teenage use.

Example 3: A gynecological vaginal lotion for regulating female vaginal microecology, adding artificial flavors vanilla tincture with artificial electrolytic acidic water concentration of 0.04% by artificial electrolytic acidic water, and adjusting the pH value to 3.5 after disinfection.

Artificial electrolytic acid water for the natural water or tap water concentration of less than 0.2% salt such as sodium chloride, the use of electric field electrolysis prepared.

Example 4: A gynecological vaginal lotion for the regulation of female vaginal micro-ecology, which is obtained by adding artificial spicy white orchid balsam, which is 0.1% by volume of artificial ionized acidic water, and adjusted to pH 3.7 after disinfection.

Artificial ionization of acidic water for natural water or tap water through the purification into the high efficiency ionization tank, so that water ionization, and then positive and negative ion water separation. Through the use of activated carbon as a filter layer, filter tap water, and then through the electrolytic cell with a membrane electrolysis, select the concentrated in the electrolytic cell anode water is artificial ionization of acidic water.

Example 5: A gynecological vaginal lotion for the regulation of female vaginal microecology, acid water prepared by mixing nanometer bubbles of hydrogen-rich water and artificial electrolytic acidic water in any proportion, said nano-bubbles rich in hydrogen acid water and artificial Electrolytic acid water is commercially available; add natural water spicy resin with a concentration of 0.5% acidic water, adjust the pH value of 3.8-5.0, for mature women; pH 4.5-5.5, for girls use.

Example 6: A gynecological vaginal lotion for the regulation of female vaginal microecology, acidic water prepared from an acidic mixture of electrolytic acidic water and artificial ionized acidic water in any ratio, said electrolyzed acidic water and artificial ionized acidic water being commercially available on the market And then add the acidic water concentration of 0.3% of the natural spice narcissus net oil, adjust the pH value of 3.8-5.0, for mature women use; pH value of 4.5-5.5, for girls use.

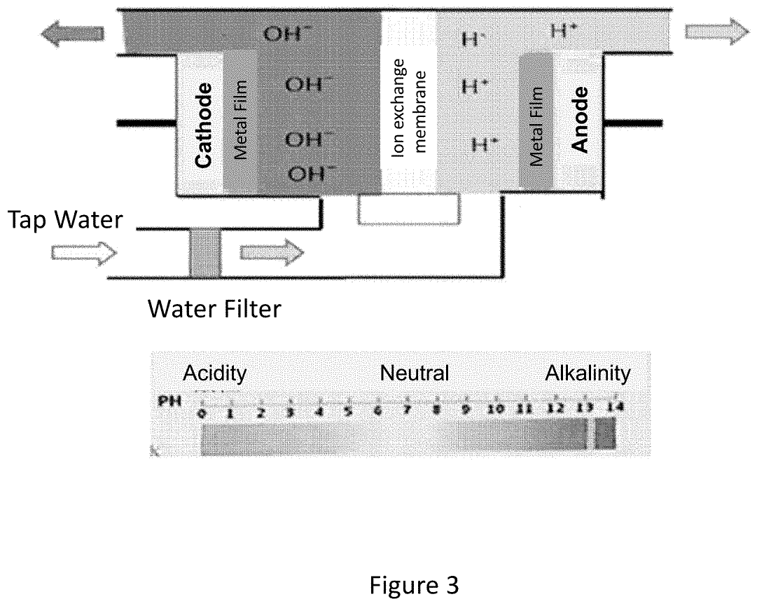

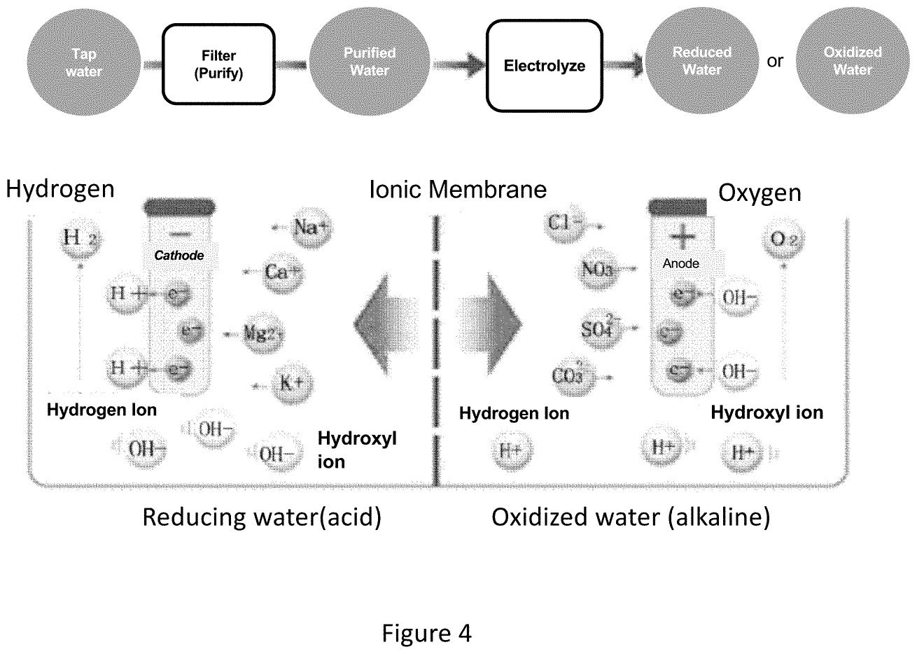

FIGS. 3 to 5 are schematic illustrations of the principles of artificial electrolytic acidic water used in the present invention. Is based on the “electrochemical” and “electrolysis” principle, the use of titanium platinum (platinum) material or other alloy material, as the electrolytic cell electrode plate, during which the separation of ceramic ion separation membrane separation and separation of electrolytic alkaline and acidic water Are made from natural water or tap water plus ionization, the pH value from the preparation of the external electric field and the size of the current, time and flow rate and the amount of additives to determine the amount. The water is filtered through a pre-filter.

Artificial ionization of acidic water preparation machine is to tap water as a water source, through the pre-filter to filter the water; and then get the water into the electrolytic tank, the separation of the film as a medium in the water to the DC voltage, the use of electrolytic plate to water decomposition , Which separates alkaline water from acidic water. In the electrolytic cell, the electrolytic cell is divided into anionic and cationic chambers by an ion film, and only ions are free to penetrate between the two chambers. Depending on the size of the industrial or home electrolysis equipment, the size of the electrolytic current ranges from a few milliamps to several amps, and the ionization electrolysis time ranges from 1 minute to 30 minutes.

The above-described embodiments illustrate embodiments of the present invention. It is to be noted, however, that the above is merely an example or an example of a process for preparing a gynecological vaginal lotion for regulating female vaginal microecology with electrolytic acidic water and a method for preparing the same. Those skilled in the art can devise many variations and optional compositions, methods and systems without departing from the spirit and scope of the invention, for example pH ranges from 4.0 to 5.5 or 3.8 to 5.0.

The appended claims are therefore intended to cover such changes and arrangements. Although the present invention has been described in detail above, the above embodiments provide one of the details of an embodiment of the present invention. A number of variations and improvements can be made without departing from the core elements of the present invention, that is: the concept of nanofibers rich in hydrogenated water and ionized acidic water can regulate the female vaginal microecological gynecological as vaginal lotion products, which belongs to the scope of protection of the present invention.

1. A gynecological vaginal lotion for the regulation of female vaginal microecology, characterized in that the gynecological vaginal lotion for the regulation of female vaginal microecology is a natural fragrance with acidic oxidation potential of water added with an acidic oxidation potential water volume concentration of 0.01% to 0.5%, adjusted to pH 3.5-5.5 after disinfection.

2. The gynecological vaginal lotion for regulating female vaginal microecology according to claim 1, characterized in that said acidic oxidation potential water is a mixture of nano-bubbles of hydrogen-rich water and electrolytic acidic water, said nano-bubbles Hydrogenated hydrogen peroxide having a hydrogen content of 1 to 50 ppm; an oxidation-reduction potential of OR of -400 mV to 1200 my; the pH of the nano-bubbles of hydrogen-rich water is 4.5 to 7.5; the pH of the electrolytic acidic water is 2.0-4.5.

3. The gynecological vaginal lotion for regulating female vaginal microecology according to claim 1, characterized in that said acidic oxidation potential water is an electrolytic acidic water having a pH of 3.8 to 5.5 and the composition thereof comprises a mass concentration of 0.1% to 0.5% of an acid salt containing a mineral acid, an effective chlorine concentration in an acid salt containing a mineral acid is at least 8 ppm, and the LD5t1 value is more than 300 mg, and the acid salt means a hydrogen atom containing a hydrogen atom capable of being substituted with a metal atom Salt, or an acid salt is an acid salt having a characteristic that the acidity is dissolved in water, i.e., a pH of less than 7.0.

4. The gynecological vaginal lotion for regulating female vaginal microecology according to claim 3, characterized in that said electrolytic acidic water is a salt of less than 0.5% by weight of natural water or tap water, and is prepared by electric field electrolysis to make.

5. The gynecological vaginal lotion for regulating female vaginal microecology according to claim 1, characterized in that said acidic oxidation potential water is a mixture of electrolytic acidic water and artificial ionized acidic water, and artificial ionized acidic water is natural water or tap water through the purification into the high efficiency ionization tank, the water ionization, and then the positive and negative ion water separation.

6. The gynecological vaginal lotion for regulating female vaginal microecology according to claim 12, characterized in that said acidic oxidation potential water is a mixture of nano-bubbles of hydrogen-rich water and artificial electrolytic acidic water.

7. The gynecological vaginal lotion for regulating vaginal microecology according to claim 1, characterized in that said acidic oxidation potential water is prepared by mixing an electrolyzed acidic water and an artificial electrolytic acidic water in an arbitrary ratio.

8. The gynecological vaginal lotion for regulating vaginal microecology according to claim 3, wherein the acid salt of the inorganic acid is an acid sodium salt of an inorganic acid, an acid potassium salt of an inorganic acid, an acid magnesium salt of an inorganic acid, and an acid barium salt of a mineral acid having a pH of 3.5 to 6.0.

9. The gynecological vaginal lotion for the regulation of female vaginal microecology according to claim 8, characterized in that the acid salt of said inorganic acid is dihydrogen disodium pyrophosphate, sodium hexametaphosphate, chlorinated Sodium, sodium dihydrogen phosphate or any one of any mixture in any ratio.

10. The gynecological vaginal lotion for the regulation of female vaginal microecology according to claim 1, characterized in that said adjusting vaginal microbial gynecological vaginal lotion has a pH of 3.8 to 5.0.

11. The gynecological vaginal lotion for the regulation of female vaginal micro-ecology according to claim 1, characterized in that said vaginal lotion has a pH of 4.5 to 5.5.

12. The gynecological vaginal lotion for regulating female vaginal micro-ecology according to claim 1, characterized in that the natural fragrance is a plant natural fragrance.
